(19) United States
(12) Patent Application Publication
(10) Pub. No.: US 2011/0052713 A1
(43) Pub. Date: Mar. 3, 2011

(54) REPAIRING DAMAGED NERVOUS SYSTEM TISSUE WITH NANOPARTICLES

(76) Inventors: Youngnam Cho, West Layfyette, IN (US); Riyi Shi, West Lafayette, IN (US); Albena Ivanslevic, West Lafayette, IN (US); Richard Borgens, City Delphi, IN (US)

(21) Appl. No.: 12/863,357

(22) PCT Filed: Jan. 16, 2009
(86) PCT No.: PCT/US2009/031274

§ 371 Date: Aug. 5, 2010

Related U.S. Application Data
(60) Provisional application No. 61/021,444, filed on Jan. 16, 2008.
(60) Provisional application No. 61/029,514, filed on Feb. 18, 2008.

(51) Int. Cl. A61K 9/14; A61K 33/00; A61K 31/722; A61K 33/42; A61P 25/00

(52) U.S. Cl. 424/493; 424/724; 514/55; 424/606

(57) ABSTRACT

The present application relates to reduction of acrolein-mediated cell death following neural insult. According to at least one embodiment, chitosan is utilized as a membrane fusogen to restore cell function. According to at least one other embodiment, chitosan or silica is used to create a non-toxic polymer surfaced microcolloid (PSM). PSMs were found to preferentially target the damaged nerve tissues; to restore conduction of nerve impulses; to seal/restore nerve fiber membranes; and to reduce to baseline the efflux of a large intracellular enzyme. PSMs are further used as a drug delivery vehicle for acrolein scavengers including hydralazine.

### PRIORITY

This application is a nationalization of a Patent Cooperation Treaty application PCT/US09/31274 entitled Repairing Damaged Nervous System Tissue With Nanoparticles, filed with the U.S. Receiving Office on Jan. 16, 2009, which claims priority to U.S. Provisional Patent Application Ser. No. 61/021,444, entitled Repairing Damaged Nervous System Tissue With Nanoparticles, filed Jan. 16, 2008, the contents of which are incorporated by reference herein.

### BACKGROUND

The biological basis for functional loss after spinal cord injury, whether the injury is acute, chronic, or via a neurodegenerative disorder is the elimination of nerve impulse transmission “up and down” the spinal cord. The basis for a partial functional recovery, independent of how old the injury is, is the restoration of such nerve impulses. Currently, there is no medical therapy for severe spinal cord or brain injury that can restore behavioral loss in the chronic condition, or rapidly repair the membranes of damaged nerve cells in the acute stage of the injury.

Mechanical damage to the nervous system of mammals results in sometimes irreversible functional deficits. Most functional deficits associated with trauma to both the Peripheral Nervous System (PNS) or Central Nervous System (CNS) result from damage to the nerve fiber or axon, blocking the flow of nerve impulse traffic along the nerve fiber. This may be due to a physical discontinuity in the cable produced by axotomy. The blockage may also occur where the membrane no longer functions as an ionic fence, and/or becomes focally demyelinated In either case, functional deficits occur because of the break in nerve impulse conduction. Even the severe behavioral deficits associated with spinal cord injury is now understood to be largely due to the initial mechanical damage to white matter. Delayed but progressive episodes of so-called “secondary injury” subsequently enlarge the lesion leading to the typical clinical picture of a cavitated contused spinal cord, and intractable behavioral loss.

The popular notion that the spinal cord is “severed” in acute injuries is largely incorrect, as true anatomical transection of the spinal cord is quite rare in human injuries. After an acute injury, there is a variable amount—or “rind”—of spinal cord white matter left intact. However, this region of anatomically intact nerve fibers does not function. In particular, this local region (usually less than 1 vertebral segment in extent) does not conduct nerve impulses through the region of damage. This is believed to be due to demyelination, as well as other factors. The loss, or the reduced thickness of myelin, which insulates the nerve process, causes conduction blockage at the Nodes of Ranvier. This is because so-called “voltage gated” fast potassium channels are localized at paranodal regions in myelinated nerve fibers underneath an insulating layer of myelin. When myelin retracts or is lost after injury or disease, the clusters of potassium channels are exposed to extracellular fluids and are also deprived of their electrical insulation. Potassium loss though these naked channels both increases the extracellular concentration of potassium, and helps extinguish a nerve impulse (actually a depolarization of this local nerve membrane). Indeed, it is well known that the extracellular microenvironment near a spinal injury is rich in potassium, which by itself dampens the ability of nervous tissue to function normally.

Moreover, the loss of the electrical insulating capacity of myelin facilitates short circuit potassium current that aids in extinguishing the nerve impulse before it can begin to cross the nodal region. Drugs that block this exodus of potassium from inside the nerve fiber to the outside milieu (so called channel blockers) are believed to be the biological basis for the restoration of action potential (or nerve impulse) conduction through spinal lesions associated with variable recoveries of functions in human patients. The only drug of this type, 4-Aminopyridine (the “time release” form of the drug is called Fampridine), has shown promise in restoring nerve function in paralyzed persons. However, clinically meaningful recoveries of function only occur in about 30% of the treated population, and in the balance, these recoveries are associated with numerous unwanted side effects that occur at the concentrations of the drug required. Such unacceptable side effects include dizziness mid loss of balance at one end of a scale—to the possibility of seizures at the other.

As another means for treating cell membrane damage, hydrophilic polymers such as polyethylene glycol (“PEG”) have been used to fuse cell membranes. Fusion of defective cell membranes with PEG or copolymers such as the Poloxamines has been shown to vitiate or alleviate conditions as diverse as burns, myonecrosis, and testicular reperfusion injury in animal models. The molecular mechanisms of membrane fusion/reassembly by polymers are still investigated using model membranes. Importantly, polymer administration can be completely safe for Human application, and it has been shown that injection of ˜2000-3000 MW PEG or Poloxamer 1100 in animals can produce anatomical sealing of damaged neuron membranes and restore their conduction properties in hours to days. Anatomical repair and functional recovery occurred in both spinal cord injured and brain injured guinea pigs and rats.

This work has progressed through clinical trials in canine paraplegia. However, it is not currently known how to increase either the MW of the polymer or its aqueous concentration in an effort to widen the therapeutic window and behavioral recovery after CNS damage. This is due to the fact that at higher MWs or concentration, the increased viscosity limits the clinical utility of PEG. Conversely, lower molecular weights below ˜1000 KD carry the risk of metabolized subunits producing ethylene glycol poisoning. As such, an alternative to the use of PEG to heal damaged nervous system membranes or hydralazine to increase nerve function after an injury would be greatly appreciated.

### BRIEF DESCRIPTION OF THE DRAWINGS

FIGS. 1A, 1B, 1C, and 1D are a series of diagrams visually depicting the process of producing a polymer surfaced microcolloid according to one aspect of the present application.

FIG. 1E is a transmission electron microscope image of a Tetramethyl Orthosilicate (TEOS) surfaced Silica base produced according to at least one method of the present application.

FIG. 1F, is a transmission electron microscope image of a Tetramethyl Orthosilicate (TEOS) surfaced Silica base functionalized with PEG produced according to at least one method of the present application.

FIG. 2A is a perspective rendering of a double sucrose gap chamber utilized to determine the efficacy of a polymer surfaced microcolloid according to one aspect of the present application.

FIG. 2B is a graph showing the CAP Amplitude through a crushed guinea pig spinal cord and comparing the CAP Amplitude when a polymer surfaced microcolloid including polyethylene glycol is utilized, versus when no treatment is made, versus utilizing a microcolloid having no polymer modified surface.

FIG. 2C is a bar graph showing the potential amplitude through a crushed guinea pig spinal cord and comparing the CAP Amplitude when a polymer surfaced microcolloid including polyethylene glycol is utilized, versus when no treatment is made, versus utilizing a microcolloid having no polymer modified surface.

FIGS. 3A and 3B are cross sections of a spinal cord shown in darkfield fluorescence, wherein FIG. 3A shows a damaged and untreated spinal cord while FIG. 3B shows an injured spinal cord treated with a polymer surfaced microcolloid according to one aspect of the present application.

FIG. 3C is a bar graph showing the uptake of dye in an injured spinal cord versus the uptake of dye in an injured spinal cord treated with a polymer surfaced microcolloid according to one aspect of the present application.

FIGS. 3D through F are cross sections of injured a spinal cord treated with horse radishperoxidase.

FIG. 3G is a bar graph showing the density of axons permeable to horse radishperoxidase in an injured spinal cord versus the uptake of dye in an injured spinal cord treated with a polymer surfaced microcolloid according to one aspect of the present application.

FIG. 3H is a bar graph showing LDH release in an injured spinal cord versus the uptake of dye in an injured spinal cord treated with a polymer surfaced microcolloid according to one aspect of the present application.

FIG. 4 shows cross sections of a spinal cord shown in darkfield fluorescence, wherein a damaged and untreated spinal cord is compared to an injured spinal cord treated with a polymer surfaced microcolloid according to one aspect of the present application.

### DETAILED DESCRIPTION

The present application relates to colloidal compositions for treatment of peripheral nervous system and central nervous system injuries, insults and diseases, and methods of producing such compositions.

### Polymer Surfaced Microcolloids and Their Manufacture

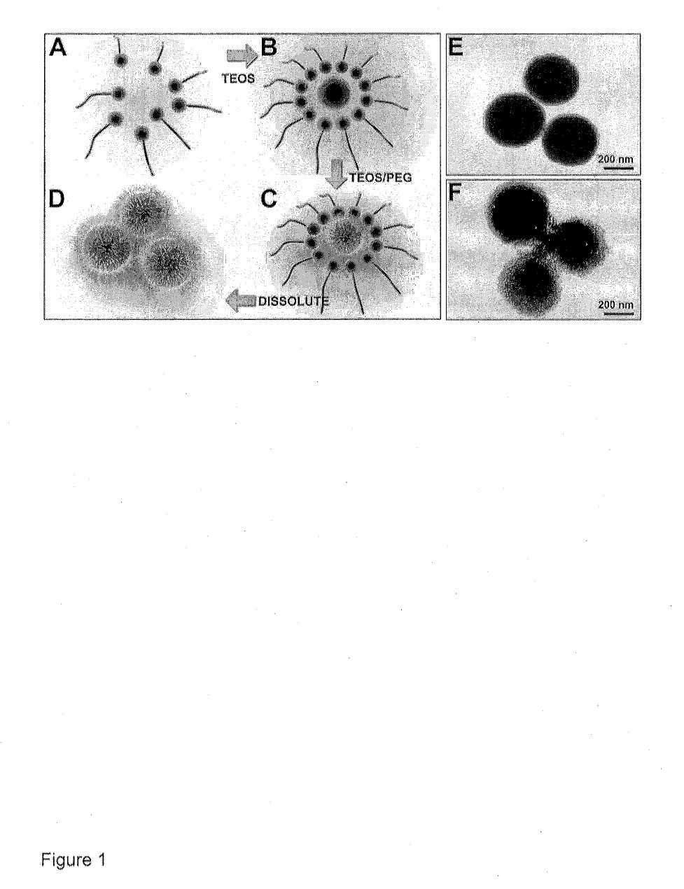

According to at least one embodiment, a non-toxic polymer surfaced microcolloid (“PSM”), such as a polymer surfaced, silica based microcolloid (“PSMC”) is created as shown in a rendering in FIGS. 1 A-C and utilizing techniques for creating mircocolloids as described in Lindberg, R.; Sjoblom, J.; Sundholm, G., Colloids Surf A. 99: 79-88 (1995); Santra, S. et al,, Anal. Chem. 73:4988-4993 (2001); Bagwe, R. P.; Hilliard, L. R.; Tan, W., Langmuir 22: 4357-4362 (2006). As shown in FIGS. 1A-1D, polyethylene glycol (“PEG”) is combined with tetramethyl orthosilicate (“TEOS”) by combining water-in-oil (“W/O”) or reverse microemulsion with sol-gel technology. The process is optionally performed at about room temperature by hydrolysis of metal-organic compounds followed by post coating with PEG.

According to at least one embodiment, the microemulsion solution is comprised of surfactant, cosurfactant, at least one organic solvent, water, and aqueous ammonia. It will be appreciated that aqueous ammonia serves as both a reactant and a catalyst, wherein the water is the reactant and the NH₃ is the catalyst for the hydrolysis of TEOS. Further, it will be appreciated that the surfactant, cosurfactant, at least one organic solvent, water, and aqueous ammonia can be mixed in varying proportions as currently practiced in reverse microemulsion systems. The microemulsion solution is allowed to react for approximately twenty-four (24) hours, prior to addition of TEOS and PEG (KW. 2000) at concentrations determined by the target size of the resultant PSMC. After another approximately twenty-four (24) hours, the resultant particles are released from the microemulsion by the addition of ethanol or another appropriate solvent. In this instance, the hydrogen bond formed from the interaction of ether oxygen with PEG and the residual silanol groups (Si—OH) in the silica network facilitates the formation of colloidal silica particles during polycondensation. Transmission electron microscopy (“TEM”) of the resultant particles indicates that the PSMC resulting from this method are regular in size, shape, and structure, as shown in FIG. 1E. In this instance, it can be seen that the resultant individual PSMC's are approximately 400 nm in diameter. In addition, as shown in FIG. 1F by the blurred edges of the individual PSMC's in the TEM images, the PEG polymers are coated on the surface of the particles by polycondensation with PEG.

The resultant PSMC's were fully characterized and their size distribution, chemical functionality, and type of ligand bonding was confirmed. TEM images were used to determine the regularity of size, shape, and structure of the resultant PSMC's of varying diameter according to at least one embodiment. As can be seen, PSMC's of approximately 400 nm, 200 nm, and 20 nm were produced, with the approximate percentage of particles at each size in the mixture being shown in the bar graph below each image. In addition, the Fourier Transformed Infrared (“FT-IR”) spectra of bare silica and PEG-modified silica was analyzed. When compared to bare silica, PEGs-modified silica exhibit characteristic peaks at 2900˜3000 cm−1, indicating the presence of C—H groups. The absorption peaks at 3200˜3600 cm−1 are assigned to the Si—OH bond stretching. The bands in the low-frequency region (600˜1500 cm−1) correspond to the silica network: asymmetric and symmetric stretching of CH2, CO—C, and Si—O—Si bonds.

In addition, x-ray photoelectric spectroscopy (“XPS”) was used to monitor the chemical composition of the synthesized colloids. The C 1s and O 1s peaks were examined because these two components allow the evaluation of the chemical structures of the species present on the surface. After adsorption of PEG, silica particles exhibit two obvious peaks assigned to C—C (284.0 eV) and C—O peaks that resulted from the reaction with PEG. The relatively high intensity of the C—O peak represents the saturation of the particle surface with PEGs. The presence of monocomponent shape of O 1s spectrum further confirmed the existence of PEG on the silica surface in the form of single bonded structure C—O. The FT-IR and XPS results indicate the presence of concentrated PEG polymer chains on the surface.

It will be appreciated that other materials besides silica may be used as a base for the production of polymer surfaced microcolloids. For example, rather than utilizing TEOS as the base, chitosin may be utilized as the base, providing a biodegradable base which has a surface that can be PEG modified. In addition, the versatility of materials with inherent unique properties (optical, electrical, magnetic, and chemical) can be realized with the incorporation of a variety of biocompatible and biodegradable materials such as synthetic or natural polymers, lipids, or solid (metal, semiconductor, magnetic, or insulator) components. For example, magnetic particles may be attached to the base material, or fluorescent dyes or radiolabels may be utilized as well.

While biodegradable or organic particles may be used as a base material, inorganic particles such as the silica base disclosed above may be utilized. For purposes of the example above, silica particles were used because silica based particles have great potential to perform multifunctional activity, and silica particles exhibit intrinsic hydrophilicity, biocompatibility, and non-toxicity. In addition, inorganic cores, rather than organic such as micelles, have a longer “shelf life”.

### II. Use of Polyethylene Glycol Coated Silica Particles to Treat Injured Nerve Tissue

According to at least one embodiment, the biological activity of the PSMC's described above was tested by one or more of the following methods: (1) evaluating the loss and recovery of electrophysiological conduction of nerve impulses through isolated guinea pig spinal cord white matter, and (2) determining the ability of spinal cord tissue to reconstitute and seal damaged neuron/axon membranes against the uptake of extracellular labels or the loss of endogenous macromolecules from their cytoplasm. Each of these functional assays are mutually dependent on each other. See R. Shi, J. D. Pryor, Neuroscience 98:157-166 (2000).

While, generally, it is believed that nanomedicine tools have the potential to lead to more effective ways to treat and predict disease, it has not been previously known how the use of nanoparticles or polymer surfaced microcolloids will affect the treatment of injured nerve tissue in vivo. Downing, G., Science 310, 1132-1134 (2005); Callaway, E., Science 314:1674-1676 (2006).

### A. Example I

### Administration of PSMC to Damaged Spinal Cord

According to at least one exemplary embodiment, the administration of PSMC to severely injured mammalian spinal cord restored anatomical structure and physiological functioning. It is believed that this restoration of structure and physiological functioning is accomplished through the PSMC surface coat interaction with the damaged membranes nerve processes (axons), leading to spontaneous reassembly of the compromised membrane in the damaged area, anatomical sealing, and the immediate recovery of nerve impulse propagation along the length of adult guinea pig spinal cord white matter.

As discussed above, the creation of this PSMC was undertaken using a synthesis of colloids, functionalized for targeting nerve membrane repair using polyethylene glycol. Using a colloid based derivative, tunable concentrations of membrane-active molecules on the PSMC's surfaces permits a significantly improved ability to control concentration and density of the polymer at the specific regions of CNS damage.

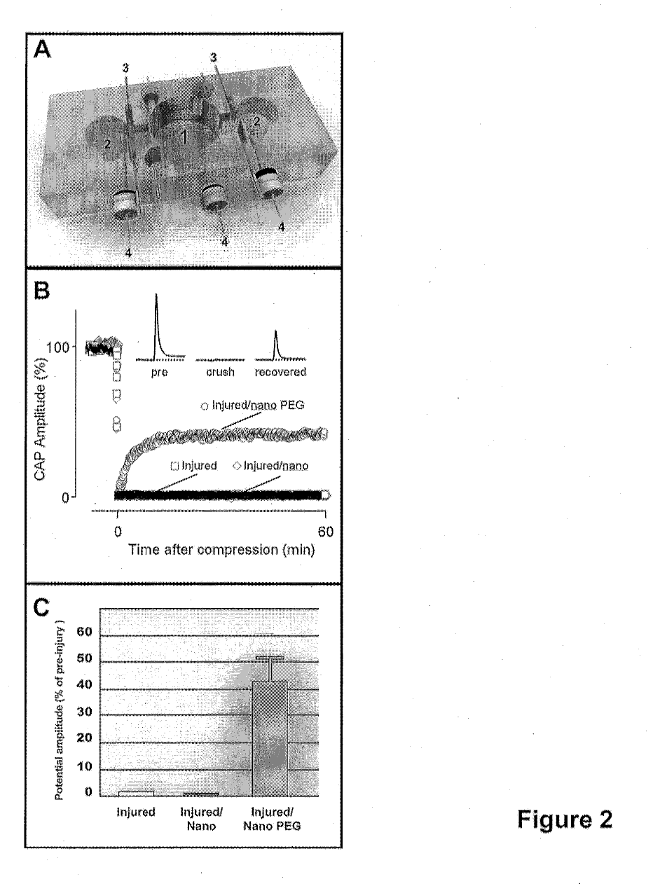

Conduction through the length of an entire guinea pig cord (˜40 mm) was studied in a double sucrose gap isolation/recording chamber discussed in R. Shi, A. R. Blight, J. Neurophysiol 76: 1572-1580 (1996). This technique provides the most sensitive and highest resolution recordings on ex vivo spinal cord or peripheral nerve currently known. Strips of isolated guinea pig ventral white matter bathed in a Physiological Kreb's solution were evaluated because this region of the cord possesses the highest density of large, fast conducting, myelinated axons. The normal characteristics of conduction were first recorded, and subsequently, the cord was crushed in the mid-thoracic region using a laboratory fashioned device. This produced a detente to standardize compression between cords. Luo, J, Borgens, R. B. Shi, R.; J Neurotrauma 21(8) 994-1007 (2004). This procedure eliminated nerve impulse conduction through the white matter as shown in FIG. 2B.

The recovery of conduction, or the lack thereof, was then recorded in: (1) control un-treated crushed cords, (2) after the addition of “control” silica particles without a PEG surface, and (3) after the addition of colloids possessing a polymeric surface coat. With only a single exception, only those spinal cord strips treated with PSMC's were electrophysiologically competent within one hour of compression injury (FIGS. 2B, 2C). Using these same techniques, spontaneous recovery of compound action potentials after compression rarely occurs—and then at times greater than one hour after injury. In one untreated (control) spinal cord out of seven tested, we observed the appearance of a low amplitude, weak, and unstable compound action potential (“CAP”).

Compound Action Potentials (CAPS) are stimulated on end of the white matter strip, and recorded at the other. As shown in FIG. 2B, the CAP of three spinal cord records are shown from left to right. The first (labeled “pre”) shows a typical CAP pre-injury, followed by a second CAP (labeled “crush”), its immediate elimination after compression in the center of the strip. The third record (labeled “recovered”) shows the recovery of the CAP, 60 min after topical application of 5 mM solution of PSMCs in distilled water. Crushed cords do not spontaneously recover CAPs at this time. Below these records, graphs of CAP amplitude vs. time show the responses of individual strips: injured but not treated, and PMSC-treated. The average responses and their SEMs are shown for of all 21 spinal cords in the bottom histogram (C., 7 cords in each group). The absence of any measurable CAP recovery is shown for silica treated, while a miniscule recovery is shown for the uninjured cords due to one outlier (see text). Note the striking recovery of CAP propagation following treatment with the PEG Microspheres. (P≦0.001).

In all tissue treated with PSMCs, statistically significant CAPS of marked amplitudes reappeared within 5 minutes. These initial CAPS then increased in magnitude over the one hour of observation. (FIGS. 2 B and C). Uncoated microcolloids had no effect whatsoever on electrophysiological conduction. Anatomical sealing in whole spinal cords isolated to Krebs's solution was tested by application of the fluorescent label tetramethyl rhodamine dextran (TMR; 10 kD) to the bathing solution after complete transection of the whole spinal cord. Turning to FIG. 4, numerous photographs of the TMR treated PSMC's are shown, comparing uninjured spinal cord cross sections with injured spinal cord cross sections treated with TMR treated PSMC's.

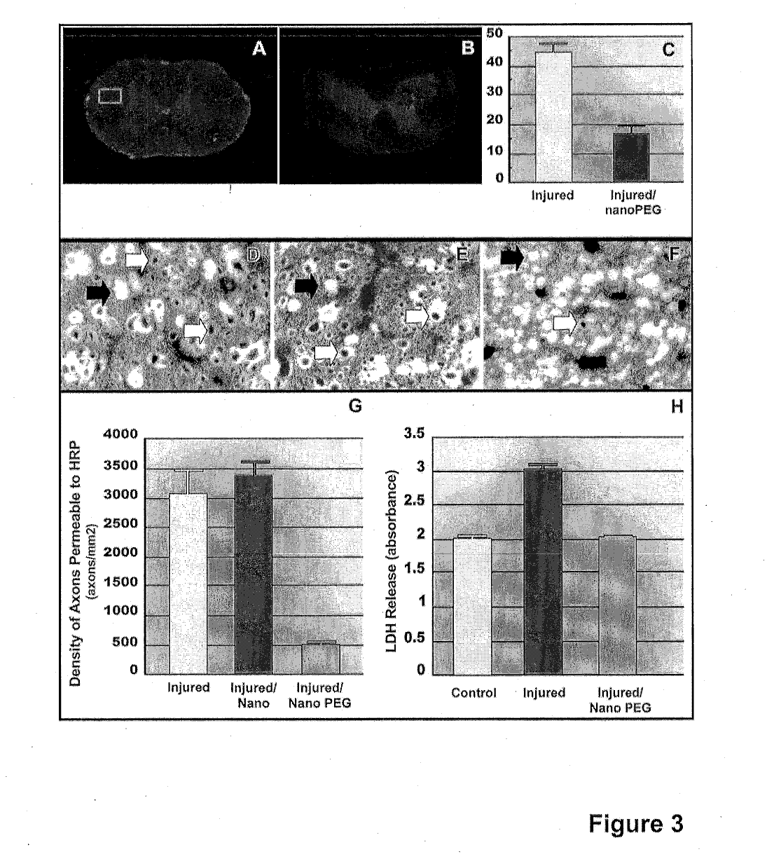

This label is imbibed into damaged neurons and their processes, allowing the existence of the damage to be seen. Since the behavioral loss in animals and man after spinal cord injury (“SCI”) is due to the failure of conduction though white matter—and not the loss of neurons only white matter was evaluated in these studies. In transected spinal cords, the labeling of white matter was quantified using NIH Image™ software (FIGS. 3 A and B). Untreated white matter of all severed cords was labeled, while the labeling of PSMC treated spinal cords was barely detectable. Quantification of fluorescence (minus background) statistically confirmed what was easily observed in paired treated and un-treated cords (FIG. 3 C). This result suggested the recovery of electrophysiological conduction was tied to rapid sealing and sparing of the cell processes of white matter. This is an extreme “proof of concept” test as complete transection of cords in clinical injuries is uncommon

Turning now to FIGS. 3A and 3B, cross sections of spinal cord are shown in darkfield fluorescence illumination. According to one exemplary embodiment, cords were exposed to tetramethyl rhodamine dextran (“TMR”; 1000 Daltons) for 10 minutes prior to fixation and 4 hours before sectioning (60 urn on a freezing microtome). Cords were exposed to a solution of PSMCs after a complete transection, but prior to staining with TMR for 15 min. Untreated Cords were treated similarly. FIG. 3A shows an injured untreated cord. Note the fluorescence in white matter associated with TMR uptake. This labeling was strikingly and statistically reduced by PSMC exposure (the yellow inset shows the sample region for each pair of cords using NIH Image software). A total of 14 cords were used, treated as 7 pairs; one experimental (PSMC treated) and one control untreated). For every cord, three records of intensity within the sample region were recorded within both left and right funuclii and the ventral funuclus.

Approximately 150 ˜m outside of the cord, and directly adjacent the original sample region, three similar records were obtained to record background fluorescence. The difference between these is displayed in Arbitrary Units (1-250 scale). As shown in FIG. 3D, a larger marker for membrane compromise, Horse Radishperoxidase, (“HRP”; 40,000 kD), was significantly imbibed into large numbers of crushed axons. Each test solution was applied for ˜2 minutes, and the cord removed from the test chamber and fixed within 20 minutes. The white “halo” around each darkly label axon cross section is the myelin sheath as shown in FIGS. 3E and 3F. In FIG. 3E, a similar level of labeling is shown after treatment with uncoated microcolloids. In FIG. 3F, PSMCs produced a striking and statistically significant reduction in membrane damage as revealed by HRP exclusion in FIG. 3G. Another indicator of membrane damage is the leakage of the enzyme, Lactic Dehydrogenase (LDH; 144 KD) into the extracellular milieu. Testing is accomplished by incubating crushed spinal cords or Control spinal cords in Kreb's solution for about 1 hour, collecting the supernatant, and determining LDH concentration by spectrophotometric techniques. FIG. 3H shows the background level of LDH loss (enhanced by dissection, handling, and natural extrusion of the enzyme). It will be appreciated that crush injury significantly increased LDH in the supernatant, while treatment with PSMCs returned the level of LDH loss to precrush levels.

As discussed above, the result of the “dye exclusion” test (Shi, R., Borgens R. B.; J. Neurocytology 29:633-643 (2000)) was further confirmed by PSMC-mediated exclusion of a larger label, horseradish peroxidase (“HRP”; ˜41 kD). HRP, when added to the bathing media of injured spinal cords heavily labeled damaged axons, reveals a dark brown/black axonal cross section framed by the wrapping of unstained myelin (FIGS. 3 D-F). This uptake was significantly reduced after treatment with PSMCs. Similarly, the normal background loss of the cytoplasmic enzyme Lactic Dehydrogensase (LDH; 144 kD) to the extracellular milieu is significantly increased after damage to the axolemma. Treatment with PSMCs after injury substantially reduced this efflux to normal baseline levels (FIG. 3 H).

Turning now to FIG. 5, the somatosensory evoked potential (“SSEP”) of guinea pigs were tested pre-injury, post-injury, post-injury 20 minutes post-injection of 50 nm diameter PSMC's, and post-injury 7 days post-injection of 50 nm diameter PSMC's. The traces were produced by stimulation of the Tibial nerve of the Guinea Pig hindleg—and measuring the nerve impulses (evoked potentials) as they “arrive ” later at the contralateral sensorimotor cortex of the Brain. These records were made utilizing pin electrodes for both stimulation and recording. Further utilized was a control recording procedure, where the median nerve of the forelimb is stimulated and SSEPs are recorded. Since the forelimb is rostral to the midthoracic injury, in every case a SSEP will be produced. This controls for false negative recordings, and was carried out during every recording period.

An additional property of the PSMC composites is its preferential targeting of damaged CNS. This preferential targeting was revealed by incorporating a label to the composite and comparing its distribution between crushed and undamaged spinal cord. These results provide the first evidence for substantial neuroprotection correlated to a physiological recovery in significantly damaged CNS tissue using microcolloid technology. This technique is based on the engineered surface properties of the colloid which is not typical of pharmacological covalent bonding. These colloids are unable to be metabolized (as is the case with drugs) and would be injected into the patient's bloodstream to be harmlessly passed out of the body as both native components are inert and/or nontoxic. The technology reported herein permits precise, inert and non toxic, particle fabrication possessing densities of surface active agents many orders of magnitude greater than can be achieved by conventional systemic administration. This also beneficially impacts the occurrence of concentration dependent side effects.

### B. Example II

### Synthesis and Functionalization of Mesoporous Silica Nanoparticles (MSN)

All chemicals were purchased from Sigma-Aldrich unless otherwise specified. MCM 41-type mesoporous silica nanoparticles (MSNs) were synthesized according to the procedure described by Slowing et al. First, cetyltrimethylammonium bromide (CTAB), used as a template, was dissolved in a solution of deionized water and ammonia. After stirring at 80° C. for 2 hr, tetraethyl orthosilicate (TEOS) was slowly added to the mixture. The solutions were stirred at elevated temperature for another 3 hr and then the white precipitate was collected by filtration, rinsed with water, and dried at 100° C. for 12 hr. Finally, an acidic extraction method (0.75 mL concentrated HCl/100 mL methanol solution) was performed overnight to remove the CTAB template. MSN incorporating hydralazine (MSN-Hy) was prepared by adding 20 mg of as-synthesized MSNs to 10 mL of a 50 mM hydralazine solution. The mixture was shaken at room temperature for 24 hr. The product was separated by centrifugation and dried in an oven overnight. The particles were further modified to covalently link PEG to MSN surface-using 3-(trimethoxysilyl) propyl aldehyde followed by coupling with PEG-NH2 (M.W. 3000).

1. Functionalization of MSNs with Drug/Polymer Conjugation

The polymer-drug conjugation on MSNs has significant advantages in that i) Hydralazine incorporated inside the channels of the silica framework can be safely delivered in the cytoplasm to scavenge reactive oxygen species (ROS) associated with acrolein, ii) the application of PEG after injury inhibit the process of necrosis occurred by acute membrane disruption and facilitate the integrity of cell membrane, thus eventually maintain the intracellular level of ions, and iii) the particles with ˜100 nm in a diameter are able to be efficiently internalized into cytoplasm by endocytosis to directly interact with cell compartments. Well-ordered internal structure of MSN served as a template for hydralazine adsorption by favorable electrostatic interaction between free silanol groups on the wall of pore and positively charged amine groups of drug. The successful encapsulation of hydralazine into the surface of the pores was confirmed by TEM, N₂ adsorption, XRD, UV spectroscopy, and FT-IR. Several MSNs were synthesized, including MSNs loaded hydralazine (MSN-Hy), MSNs functionalized with PEG (MSN-PEG), and MSNs with hydralazine encapsulation and PEG coating (MSN-Hy-PEG), respectively. CTAB-removed MSNs exhibit uniformity in size with regular spheres and well-defined hexagonal array. The TEM image of MSN-Hy displays the characteristic pore filling represented by dots and indicates the distribution of hydralazine both on and in the silica framework. The as-synthesized MSNs were further modified to covalently link PEG to silica surfaces, resulting in interrupting the porous structure of MSN by bulky polymer. In addition, MSN-Hy has further undergone the functionalization with PEG to the surfaces of silica. The physical properties of as-synthesized MSN and modified MSN were investigated by N₂ adsorption/desorption isotherm. The curves from MSNs and MSN-Hy exhibit no hydrolysis loop, which represents stable mesoporous features. As-synthesized MSN shows 1043 m²/g of BET surface area, 0.83 cm³/g of total pore volume, and 2.75 nm of pore diameter, respectively. However the uptake of hydralazine causes the decrease in surface area, total pore volume, and pore diameter significantly, indicating the pore filling with drug. The covalent cross-linking with PEG results in low surface area, pore volume, and high pore diameter, which becomes an obstacle for further coupling with drug due to the steric hindrance of PEG with large molecular weight (M.W. 3000). In case of MSN-Hy-PEG, it is anticipated that the particles would possess hydralazine core and PEG modified silica structure, which can be confirmed by obvious decrease of BET surface area and pore volume compared with those of MSNs. The loading degree of hydralazine corresponded to 30.1% of MSN-Hy and 23.1% of MSN-Hy-PEG, respectively. The further modification of MSN-Hy with PEG would attribute to the lower hydralazine incorporation as a consequence of some loss of hydralazine entrapped inside the pore through two-step PEG modifications. The powder x-ray diffraction (XRD) presents specific information regarding the change of internal structure before and after loading with hydralazine and/or coating with PEG. As-synthesized MSNs showed a strong reflection at (100) and (110). However, after functionalization, mesopores still displayed their inherent hexagonal array but the intensity of scattering was decreased in an obvious fashion. This different behavior is attributed to the pore filling effect, which is consistent with other studies. As an ideal delivery system, a drug has to be localized specifically and directly to its intended target. Compared with intravenous administration of free drug/polymer, the attraction of MSNs-based system is the capability of nanoparticles to cross membrane barriers, especially with specificity. The cellular uptake of MSNs was observed by TEM, indicate that the particles entered into the cell by endocytosis and accumulated in the cytoplasm. Nanoparticle-based drug delivery not only protects drugs from denaturation and degradation, but also maintains the activity of the drug and enhances the bioavailability through uptake. The release behavior of hydralazine from MSNs in Krebs' solution over 5 days is as follows: 80% of the adsorbed hydralazine was released from MSN within 1 day while the MSN coated with PEG delays hydralazine release in an obvious way. The slower release rate could be explained by the presence of PEG covering around the external surface of silica particles. When PEG is conjugated to the MSN, the bulkiness of the PEG polymer would enhance the stability of the encapsulated drug and prevent release. This suggests that the release behavior of a drug would be controllable by varying the type and concentration of the polymer agent.

2. Understanding of Injury and Cytotoxicity of Acrolein

Mechanical damage to cell membranes, referred to as the ‘primary injury’, produces a break in the semipermeable membrane causing a loss in ionic sealing. This results in the poor regulation of ionic species crossing or being transported across the membrane. Eventually ions, especially Ca²⁺, are able to move freely between intracellular and extracellular compartments causing significant cell pathology. Such ionic derangement causes the progressive destruction of cytoarchitecture (through climbing concentrations of free Ca⁺⁺) and eventually the cell body through a cascade of pathophysiological processes. The membrane endures the collapse of mitochondrial anatomy and physiology. Aberrant oxidative metabolism by then compromised mitochondria accelerates the production of free radicals including super oxide (O₂⁻), hydroxyl ions (OH), and hydrogen peroxide (H₂O₂). The overproduction of such “anti-oxidants” leads to further deterioration of the integrity of cell membranes by releasing free fatty acids. Continuing peroxidation of free membrane lipids results in the production of endogenous aldehydes into the cytosol—all of which are toxic—principally acrolein. Such endogenous toxins can pass the intact membrane freely, thus the extracellular concentration of these cellular poisons increases, and as cells die, even more acrolein is released—inducing the progressive destruction of nearby “healthy” cells. These independent and overlapping chemistries, and the progressive destruction of tissue they cause, are referred to as secondary injury.

3. Cell Viability Test by MTT and LDH Assay

Exposure to 100 μM acrolein can induce the death of >80% of a population of PC 12 Cells by a few hours, and 100% by 8-12 hours of observation [3]. In this study, we compared the effectiveness of hydralazine and MSNs functionalized with hydralazine and PEG to rescue injured cells from acrolein toxicity using the MTT and LDH assays. First, we observed the responses of entire cell population in seven different groups: 1) a control group, where cells were cultured in HBSS and “treated” with HBSS as the vehicle instead of acrolein, (2) a 100 μM acrolein-exposed group, (3) an 100 μM acrolein-exposed and 500 μM hydralazine-treated group, were hydralazine was added in the cell medium within 15 min delay after the exposure to acrolein, (4) an acrolein-exposed and post-treated with MSNs with 15 min delay after the exposure to acrolein, (5) an acrolein-exposed and post-treated with MSN-PEG group within 15 min delay after the exposure of acrolein, (6) an acrolein-exposed and post-treated with MSN-Hy group with 15 min delay after the exposure to acrolein, and (7) an acrolein-exposed and post-treated with MSN-Hy-PEG group with 15 min delay after the exposure to acrolein. Typically, populations of cells with acrolein-mediated damage show a significant reduction in MTT activity due to the collapse of mitochondria function. However hydralazine or MSNs functionalized with hydralazine recover from mitochondrial injury and abnormal oxidative metabolism. In PC 12 cells, the exposure of 100 μM acrolein decreased the absorbance to 11.0±10.2% of controls values (100%, P<0.001, n=5) after 5 hr. In cells treated with acrolein and hydralazine, this reduction was only 53.8±18.6% of controls. However, the cells treated with MSN-Hy and MSN-Hy-PEG displayed significant enhancement in. cell viability recovering to 85.5±16.6%, 59.8±29.6% after 5 hr, respectively. The significant performance of MSN-Hy-PEG in the MTT assay may be due to the presence of PEG on exterior surfaces of MSNs, where PEG could seal against the back-diffusion of hydralazine from the cytosol. According to the hydralazine release profile, the amount of hydralazine escaping from incubation during 5 hr was approximately 42% from MSN-Hy and 26% MSN-Hy-PEG, respectively. This result suggests that increasing the incubation time would enhance the adsorption of hydralazine into cell, thus increasing its effectiveness. On the other hand, cultures treated with MSNs and MSN-PEG did not exhibit a significant increase of MTT activity. This result is consistent with previous report, where secondary injury associated with acrolein toxicity can not be protected with the immediate application of PEG because PEG must gain access to the cytoplasm to reduce the concentration of ROS—and early after injury the membrane suffering from acrolein exposure is not compromised sufficiently to allow PEG's entry.

LDH results were also consistent with the MTT assays. After exposure to 100 μM acrolein LDH release increased to 168±15.4% of control values. After exposure to acrolein, treatment with Hydralazine reduced LDH release to 120±10% of control values (P>0.05) whereas MSN-Hy reduced this amount to 107±20% of control values (P<0.05). Significantly, MSNs coated with PEG, such as MSN-PEG and MSN-Hy-PEG, reduced LDH release to a level even below that of controls (untreated cells; 100±2% of control values), to 84±31% (P<0.001) and 90±39% of control values (P<0.001), respectively. These data indicate a more complete membrane seal by PEG concentrated on the surface of particles. Finally, “as-synthesized MSNs” as a formal control treatment was completely unable to protect cells from acrolein treatment—showing values similar to that of simple acrolein challenge (17±10%, (P<0.001)).

4. Effect of Acrolein and Functionalized MSNs on GSH Depletion and Intracellular ATP

Intracellular ATP levels are an excellent indicator of impairment of mitochondria function since continuous depletion of ATP directly results in a decrease in energy generation, ATP-mediated cell signal transduction, and may consequently induces cell death. The mechanism by acrolein inhibits mitochondria function is still not completely understood but there are two likely scenarios: i) acrolein can form Michael adducts with mitochondrial proteins and ii) acrolein prevents the coupling of oxidative phosphorylation and ATP production. Here, ATP levels are expressed as the emitted luminescence in the control group vs. experimental groups. As expected, acrolein treatment of PC 12 cells caused a dramatic decline in the intracellular ATP level (to 9±4% of control values (P<0.001)). However, once cells were exposed to hydralazine (15 min delay) after application of acrolein, ATP levels were enhanced significantly (55±8% of control values (P<0.05)). The post-treatment with MSN-Hy and MSN-Hy-PEG even more significantly enhanced ATP levels (93±5% and 91±1.6% (P>0.05), respectively). In contrast, MSNs that were not loaded with hydralazine, such as MSNs and MSN-PEG, did not show significant acrolein scavenging capability, correspondingly the concentration of ATP after these attempts was only 39±10.6% and 30±9.9% of control values, respectively (P<0.01). It is noteworthy that MSN itself is not an efficient scavenger of acrolein and intracellular ATP level is linearly related to the concentration of hydralazine loaded in the MSNs.

Inactivation of GSH function induces oxidative stress and directly facilitates the action of acrolein by increasing free radicals and lipid peroxidation unabated, GSH levels normally restrict these biochemistries. Therefore, cells can be partially protected from apoptosis and necrosis by maintaining intracellular levels of GSH. The measurement of the intracellular level of GSH was dependent on the degree of affinity between GSH and MCB, which is expressed as the percentage of fluorescence intensity of the thiol-bound MCB dye. Intracellular GSH levels are significantly decreased after exposure to acrolein, even at very low concentration (10 μM). This sensitivity is due to acrolein rapidly interacting with GSH by forming a glutathion-acrolein adduct. Exposure of acrolein, significant decreased the intracellular GSH level (38±9.9% (P<0.01)) as expected in PC 12 cells. After the application of hydralazine, GSH level were significantly increased (as a function of MCB fluorescence (70±20% (P<0.05)). MSNs without any modification show extraordinarily similar to values of intracellular GSH as acrolein group (52±14% vs 38.9±9.9% respectively (P<0.01)). MSNs functionalized with different species, MSN-PEG, MSN-Hy and MSN-Hy-PEG, all improved the support of GSH levels after exposure, to 66±11% (P<0.05), 70±13% (P<0.05) and 69±12% (P<0.05), respectively.

III. Chitosan Use in a Neuron Treatment

Acrolein is the strongest eletrophile of the reactive α, β-unsaturated aldehydes formed during lipid peroxidation induced by oxidants and oxidative stress. Acrolein, produced by various and different insults to cells, causes a diverse range of pathological biological cascades in addition to its well known ability to covalently crosslink biomolecules. It attacks the nucleophile centers in DNA and proteins, which disrupts numerous cellular processes and eventually leads to dysfunction, damage, and death by both necrosis and apoptosis. Acrolein production and accumulation is associated with oxidative stress related diseases including diabetic kidney disease, Alzheimer's disease (AD), Parkinson's disease (PD), ischemia-reperfusion injury, mechanical trauma, inflammation, and atherosclerosis. The use of an acrolein scavenger after a neural insult has occurred reduces the cascading damage that typically follows a neural insult, and hydralazine is known to be capable of inhibiting or reducing acrolein-induced damage. However, since hydralazine's principle activity is to reduce blood pressure as a common anti-hypertension drug, use in hypotensive trauma victims makes this compound dangerous for use in some patients, particularly in high concentrations. Further, while PEG can be used as a membrane fusogen, PEG and its derivative have preferable molecular weight or concentration ranges, which significantly narrows the therapeutic window, and possibly narrows its efficacy or increased the possibility of side effects in clinical trials. Therefore, according to at least one embodiment, Applicants utilize chitosan as a membrane fusogen as a replacement for PEG; and in at least one other embodiment utilize chitosan as a delivery vehicle to ameliorate the damaging effects of acrolein exposure.

A. Chitosan as a Hydralazine Delivery Vehicle for Targeting Damaged Nerves and Fusing with Damaged Membranes

In one exemplary embodiment, hydralazine-loaded chitosan nanoparticles are prepared using different types of polyanions as discussed herein and characterized for particle size, morphology, zeta potential value, and the efficiency of hydralazine entrapment and release. In a first example, hydralazine-loaded chitosan nanoparticles ranged in size from 300 nm to 350 nm in diameter, and with a tunable surface charge. The evaluation of bioactivities of chitosan nanoparticles using an in-vitro model of acrolein-mediated cell injury suggests that such chitosan nanoparticle-based systems demonstrate its capability as a novel therapy by effectively, and initially, reducing the loss of membrane integrity, and secondarily oxidative stress, lipid peroxidation, and necrosis in disorders such as spinal cord and brain injury as well as neurodegenerative disorders.

1. Preparation of Chitosan Nanoparticles: Chi-DS and Chi-TPP

Chitosan with 85% deacetylation degree and of medium weight (Chi, M.W. 200,000 Da), dextran sulfate (DS, M.W. 9,000˜20,000 Da), and sodium tripolyphosphate (TPP, M.W. 367.8 Da) were purchased from Fluka/Sigma-Aldrich. Two kinds of chitosan particles were synthesized: Chi-DS and Chi-TPP. Briefly, Chi-DS was prepared by complexation of Chi and DS, where chitosan was dissolved at 0.10% (w/v) with a 1% aqueous acetic acid solution while DS was prepared in deionized water at the concentration of 0.5 mg/ml. Equivalent volumes of chitosan and the DS solution were mixed by magnetic stirring at room temperature. Once the nanoparticle suspension started to form, the mixture was stirred for another 20 min. The formation of Chi-TPP nanoparticles was initiated by ionic gelation mechanism based on the interaction of cations and anions. Chi-TPP nanoparticles were formed spontaneously when equal volume of Chi (1.75 mg/ml) and TPP (2 mg/ml) solution were prepared and stirred at room temperature. Hydralazine-loaded chitosan nanoparticles were then immediately prepared by incorporating equivalent volume of a Chi acidic solution (1.75 mg/ml) and an aqueous TPP solution (2 mg/ml) or aqueous DS solution (0.5 mg/ml) containing hydralazine (1 mg/ml) while stirring with a magnetic bar.

The preparation of chitosan nanoparticles was conducted by adopting well-established protocols. The electrostatic interaction of positively charged amine moieties in hydralazine and chemically available functional groups of polyanions, such as phosphoric acid in TPP and the sulfate group in DS, is useful to facilitate the encapsulation of hydralazine inside chitosan nanoparticles. The morphological or physical phenomena of hydralazine encapsulation within nanoparticles were characterized by TEM and zeta potential/particle size analyzer as described above. In all experiments, chitosan nanoparticles were formed at an equivalent mass ratio of chitosan to polyanion due to the fact that high or low concentrations of chitosan compared to polyanions tends to decrease the encapsulation efficiency and/or promote aggregation of particles. Unloaded chitosan nanoparticles were measured to have diameters in the range of ˜250 nm˜300 nm. The incorporation of hydralazine caused a slight increase in the mean diameter of chitosan nanoparticles, resulting in an approximately 300 nm˜350 nm range in diameter. The surface charge of unloaded chitosan nanoparticles ranged from 10.78±1.54 mV to −7.16±3.69 mV for Chi-TPP and Chi-DS, respectively. The number of negatively charged groups of the polyanions, TPP and DS, was responsible for this difference, where DS (MW 9,000˜20,000 Da) would possess the predominant amount of sulfate groups per mole compared to the amounts of phosphoric acid of TPP (MW 368 Da) at experimental conditions (pH 3˜4). Positively charged hydralazine loading did slightly increase these values, corresponding to 14.51±2.58 mV and −4.84±1.38 mV for Chi-TPP/Hy and Chi-DS/Hy, respectively. Analysis of particle morphology revealed that Chi-TPP nanoparticles exhibited a well-defined spherical shape with a solid and consistent structure. On the contrary, Chi-DS nanoparticles showed a clustered spherical structure. The opposite charge of hydralazine and polyanions significantly contributes to concomitant increase in the efficiency of encapsulation, resulting in 15.8% and 23.5% for Chi-TPP/Hy and Chi-DS/Hy, respectively. It is worthy to note that hydralazine entrapment was increased by approximately 35% due to the association with DS compared to TPP. This is possibly attributed to the presence of sufficient negative charge densities in DS, which facilitated the encapsulation of appreciable quantities of hydralazine through the complexation process with chitosan.

2. Characterization of Chitosan Nanoparticles

Particle size and zeta potential measurements were carried out with a zeta-potential/particle size analyzer (Zetasizer). To begin, samples were diluted in deionized water and measured in an automatic mode. All measurements were performed in three˜five repetitions. The morphology of chitosan nanoparticles was observed by transmission electron microscopy (JEOL 2000FX).

3. Encapsulation and Release of Hydralazine from Particles

The amount of hydralazine encapsulated in the chitosan nanoparticles was measured by UV spectrometry following centrifugation of the samples at 15000 rpm for 30 min. The difference between the total amount of hydralazine used for the formation of chitosan nanoparticle loaded with hydralazine and untrapped hydralazine in the supernatant solution was calculated to assess the efficiency of encapsulation.

$$Hydralazine encapsulation efficiency = \frac{total hydralazine - free hydralazine}{total hydralazine}$$

To observe the release behavior of hydralazine from chitosan nanoparticles, a modified Krebs' solution (pH 7.2) that contained 124 mM NaCl, 2 mM KCl, 1.2 mM KH₂PO₄, 1.3 mM MgSO₄, 2 mM CaCl₂, 26 mM NaHCO₃ was used. The release of hydralazine suspended in this Krebs' solution was observed as a function of the concentration of incorporated hydralazine. The released hydralazine was extracted at a different time-interval and centrifuged to permit measurement by UV spectroscopy. The concentration was then calculated by linear equation to determine the hydralazine release curve.

4. Therapeutic Effect of Hydralazine-Loaded Chitosan Nanoparticles After Acrolein-Mediated Cell Injury

In one example, functional tests (MTT and LDH exclusion tests) were used to evaluate the response to different chitosan formulations in PC 12 cells suffering from acrolein exposure. We compared the effectiveness of these formulations using entire cell populations in six different groups: 1) a control group, (2) a 100 μM acrolein-exposed group, (3) a 100 μM acrolein-exposed and Chi-DS nanoparticle—treated group, (4) a 100 μM acrolein-exposed and Chi-DS/Hy nanoparticle—treated group, (5) a 100 μM acrolein-exposed and Chi-TPP nanoparticle—treated group, and (6) a 100 μM acrolein-exposed and Chi-TPP/Hy nanoparticle—treated group. First, we tested whether chitosan nanoparticles were capable of sufficiently reducing acrolein cytotoxicity using the MTT assay. In all experiments, the various types of nanoparticles were added to the cell medium after 15 min following the exposure to acrolein. As a consequence of acrolein exposure, approximately 98% of the cells in culture died within a couple of hours. Acrolein-induced cytotoxicity was expressed as a percentage of MTT activity, which is, as explained above, an indicator of aberrant mitochondria functioning. Thus, MTT activity subsequent to 100 acrolein exposure was decreased to 3.5±2.9% of controls values. In contrast, the treatment of poisoned cells with Chi-TPP or Chi-DS increased survival to 44.5±12.6%, and 37.8±9.6% of controls after 5 hr, respectively (P<0.01). Even more significant increase was observed after application of hydralazine-entrapped chitosan nanoparticles. Treatment with Chi-DS/Hy or Chi-TPP/Hy showed very significant increase in cell viability, corresponding to 120.5±26.6% and 108.6±17.1% of control values, respectively (P<0.001). Hydralazine-loaded chitosan nanoparticles improved cellular oxidative metabolism to the greatest extent—and even better than control value. As a complementary test, we observed the release of LDH from PC 12 cells following acrolein attack. LDH results were essentially consistent with the values obtained from MTT assays. The cells exposed to 100 μM acrolein caused the significant LDH increase, reaching 178±25.4% of control values. Conversely, LDH release was reduced to 117±18% and 115±10% of control values by post-treatment with Chi-DS or Chi-TPP, respectively (P<0.05). Remarkably, hydralazine-loaded chitosan nanoparticles, Chi-DS/Hy and Chi-TPP/Hy, exhibited even more reduction in LDH release to a level close to—or even below that of controls (untreated cells; 100±2% of control values), corresponding to 105±18% and 96±19% of control values, respectively (P<0.001). Additionally, cell mortality was examined using the live-dead cell assay. Consistent with all results described above, control cells in culture (with a characteristic 90±7% survival) dramatically fell to 30±8% at 5 hr when exposed to acrolein. Treatment of these poisoned cultures with different types of chitosan nanoparticles improved survival to 60˜70% .

5. Inhibition of Plasma Membrane Peroxidation by the Application of Hydralazine-Loaded Chitosan Nanoparticles

The addition of hydralazine-loaded chitosan nanoparticles to acrolein posoined cultures was able to inhibit or interfere with the generation of ROS and the associated process of membrane LPO. The levels of ROS increased approximately four-fold after exposure of cells to 100 μM acrolein, corresponding to 120±14% of control values (37±19%). Remarkably, the addition of chitosan nanoparticles reduced the level of ROS up to 39±14% of control values—indeed close to that of control groups (38±21%, P<0.05). Consistent with this striking result, the level of LPO was also significantly increased to 305±22% of control values after acrolein-induced injury. Also consistent with previous results, the LPO level corresponded to 91±5% (P<0.001) of control values upon treatment with Chi-DS/Hy, which was even lower than that of the control group.

### B. Chitosan as a Treatment for Nerve Injury

In addition to the above, according to at least one embodiment, chitosan alone or as a component of an injectible solution is a therapeutic agent restores critical anatomy and function when administered to injured nerve tissue, including central nervous system tissue. As used throughout this disclosure, injured nerve tissue relates to acute injury, whether mechanical or otherwise, degenerative nerve injuries, or other nerve-compromises resulting in a breach of the cell membrane, unless indicated otherwise. In such injuries, progressive destruction of cells and tissues occurs after mechanical trauma, however critical anatomy and function was restored by the injection of chitosan. Chitosan accumulation typically occurs around defected areas by hydrophobic interactions. Conversely, at intact membrane, high surface densities of lipid moieties inhibit the penetration of chitosan

Chitosan nanoparticles alone are capable of restoring cell viability by mediating the reconstruction of damaged membrane. However upon conjugation with hydralazine, its potential therapeutic effects are dramatically enhanced. Here we show the application of chitosan initially achieved neuroprotection by interfering with the generation of ROS and LPO. This is due to its membrane reconstruction properties rather than an ability to directly scavenge these toxins. Furthermore, chitosan treatment did not provide neuroprotection after exposure to acrolein—even in high concentrations. This fact supported the inclusion of hydralazine in the chitosan nanoparticle to directly provide this function.

1. In Vivo Application of Chitosan to Injured Spinal Cord

A total of 20 spinal injured guinea pigs were divided into two groups of 10 for in vivo conduction studies. The sham-treated control group received a single subcutaneous injection of lactated ringers while the experimental group received a single subcutaneous injection of a chitosan solution. Somatosensory evoked potential (SSEP) recordings were carried out on all animals before and after spinal cord injury, 1 day, 1 week, and 2 weeks post injury.

Evoked potentials from the extremities of guinea pigs were recorded arriving at the contralateral sensorimotor cortex using subdermal electrodes with a reference electrode situated in the ipsilateral pinna of the ear. Pairs of subdermal stimulating electrodes directly fired the tibial nerve of the hindlimb, or the median nerve of the forelimb (at stimuli trains of 200 repetitive stimulations at 3 Hz; 3 mA square wave at 200 μsec duration). The recording of tibial nerve evoked potentials at the brain is completely extinguished by midthoracic damage to the spinal cord, while not effecting the transmission of medial nerve potentials—whose neural circuit is “above” the level of the injury. The latter stimulation paradigm is a control for false negative tibial stimulation. Stimulation/recording and management of the electrical recordings were carried out on a Nihon Kohden Neuropak 4.

2. Resealing of Disrupted Plasma Membrane by the Application of Chitosan

According to one example, whole spinal cords were assessed by the TMR dye exclusion test and the LDH assay. The compromised plasma membrane was immersed in the solution of fluorescent label tetramethyl rhodamine dextran (TMR, 10 kD) after complete transection of the whole spinal cord. In transected spinal cords, damaged neurons were labeled and revealed a significant increase in TMR uptake compared with control (uninjured) groups. The fluorescence intensity following transection injury was significantly increased to 175±14% of control values (P<0.01), indicating permeability of the membrane to the entry of the hydrophilic TMR dye. However, TMR uptake in tissues immediately treated with chitosan after transection was reduced to 133.5±6% of control values. This reduction was not significantly different from the uninjured controls (P>0.05). The LDH leakage assay was used to further support the results of the TMR dye exclusion test. Similarly, complete transection induced significant release of intracellular lactic dehydrogensase (LDH, 144 kD). However, the loss of LDH was substantially reduced (to normal control levels) following chitosan treatment in a pH-dependent way. Protonated chitosan, at pH 3.0, significantly inhibited the release of LDH (90±16% of control values) while even deprotonated chitosan, at pH 12.0, still served as a sealent by reducing LDH efflux (97.5±15% of control values).

3. Chitosan-Induced Neuroprotection of Disrupted Plasma Membrane

In at least one exemplary embodiment, because acute damage to spinal cord results in significant oxidative stress, the levels of ROS and LPO generation as a result of the injury and after the application of chitosan were determined. Superoxide production, measured by oxidized HE fluorescence, was increased to 205±45% of control values after compression injury, showing prominent fluorescence in both grey and white matter. However, the application chitosan to plasma membranes reduced the level of ROS to 105±15% of control values at certain concentration, which was not significantly different from that of the control group. In a similar way, LPO induced by disruption of spinal cord cell membranes was monitored as a function of various concentration of chitosan. The level of LPO was significantly increased to 37.5±4.5 nmol/70 mg after spinal compression, compared to that of the control (uninjured) group (10.5±2 nmol/70 mg), indicating an almost four-fold increase. The application of 0.2% chitosan following compression injury reduced the level of LPO to 5.5±2.5 nmol/70 mg, which was even below that of control group—an approximate decrease of 80%.

4. The Targeting Ability of Chitosan Following Traumatic Spinal Cord Injury

According to at least one exemplary embodiment, intentionally crushed cords were incubated in the chitosan-FITC solution for 15 min followed by immersion in oxygenated Krebs' for an additional 30 min. According to the fluorescent intensity, crushed regions showed intense labeling in both gray and white matter. This represented an almost four-fold increase in fluorescent intensity compared to intact cords. In contrast, uninjured region obtained from at least 3˜4 vertebral segments of the injury site displayed faint labeling, confined to the lesion but not undamaged adjacent regions.

5. Chitosan Reduces the Disruption of Cell Membrane Following Spinal Cord Injury

According to at least one embodiment, chitosan can fuse and seal disrupted plasma membranes following nerve injury. As can be seen from the above examples, chitosan induces the formation of phospholipid aggregates that becomes a basis for plasma membrane fusion and sealing. The inherent non-toxicity and biocompatible nature of chitosan extends its capabilities as a vehicle for cell membrane fusion to medical applications. The recognition properties of chitosan with membrane phospholipids are thought to be governed by electrostatic interactions, hydrogen bonding, and hydrophobic forces.

The improvement in membrane permeability was examined by the commonly used, but singularly effective, label, TMR (10,000 M.W). The imbibement of the dye into the cytoplasm of damaged cells is dependent on the severity of the disruption. Typically, severely compromised tissues, both white and gray matter, are intensely fluorescent—whereas undamaged cells and tissues are not (as their membranes prevent the internalization of dye). The large surfactant aggregates produced by the application of chitosan significantly reduced membrane permeability to a hydrophilic TMR dye, indicating its capacity to seal membrane. Moreover, chitosan application results in the inhibition of leakage of the intracellular enzyme LDH in a pH-dependent manner (there is always some leakage of LDH in even control preparations because of the handeling and manipulation of the samples). Generally, the negatively charged cell membrane exhibits higher affinity for positively charged chitosan by an electrostatic interaction between chitosan's primary amine groups and the negative cell surface charge. These results are consistent with previous study where chitosan was adsorbed onto bacterial cells at low pH rather than high pH. In addition, chitosan as a gene delivery vector shows a higher transfection effect at low pH rather than that of high pH by the activity of protonated amine group below pH 7.0. While chitosan-induced LDH reduction in a concentration-dependent way shows no correlation, it does suggest the base concentration of chitosan likely saturated the region of damage to the membrane. However, chitosan effectively decreases the level of LDH leakage more significantly than PEG, independent of pH and concentration. Such membrane integrity assays with different molecular weights emphasized that chitosan is capable of sealing permeabilized membranes by repairing breaches through direct interaction with lipid bilayers.

6. Chitosan Targets Disrupted Membrane

At larger surface areas per molecule, which represents transected or crushed spinal cord membrane in our case, chitosan tends to be accumulated among phospholipid chains by hydrophobic interactions whereas at small areas per molecule, or intact membrane, high surface densities of lipid moieties excludes the penetration of chitosan, resulting in only a surface interaction. This suggests that chitosan preferentially and specifically targets injured tissues. This strengthens the hypothesis that highly packed phospholipid in well organized membrane would favor to exclude chitosan whereas defective areas characterized by disorganized phospholipids is likely to be penetrated by phospholipid chains, thus forming the combination of hydrophobic and electrostatic interaction.

7. Chitosan Blocks the Generation of ROS and LPO

As discussed previously, disruption of cell membranes not only affects the structural integrity of cell membranes—but also mitochondrial activity and overall cell vitality through a resultant cascade of pathophysiological events. Progressive catabolism of the membrane and the integrity of the entire cell likely begins with the increased concentration of cytosolic calcium (running down its electrochemical gradient) which destabilizes mitochondria (as well as other organelles and cytoarchitecture) shifting oxygen metabolism to the formation of reactive oxygen species (“ROS”). Thus, the production of ROS or so-called free radicals (such as superoxide (O₂⁻)), hydroxyl ions (OH), and hydrogen peroxide (H₂O₂), is a direct result leading to skyrocketing, secondary oxidative stress. Understandably, such endogenous and deleterious substances continue to result in progressive cell destruction via lipid peroxidation (“LPO”) and its derivatives, toxic intracellular aldehydes. Since these toxins like acrolein can pass intact healthy membranes—their intracellular concentration increases in the microenvironment permitting the poisoning of nearby undamaged cells (termed “bystander damage”). However, the application of chitosan not only contributes to the sealing of membrane permeability but also induces neuroprotection by interfering with progressive generation of ROS and LPO. However, we should point out chitosan, like PEG, is not an anti-oxidant in the chemical sense, without any intrinsic scavenging capability. Chitosan application induces the suppression of reactive oxygen species, apparently due to the restructuring of the plasma membrane—and even the mitochondrial membrane if the fusogen moves into the cytosol. This model is supported by the reduction of acrolein-mediated oxidative stress subsequent to chitosan application.

Although the embodiments have been described in considerable detail with reference to certain versions thereof, other versions are possible, including the use of other membrane fusogens or acrolein scavengers. Therefore, the spirit and scope of the appended claims should not be limited to the description of the preferred versions contained herein.

What is claimed is:

1. A method for treating injured or diseased nervous system tissue comprising:
a. providing a polymer surfaced microcolloid having a base material and a surface coating comprising a polymer; and
b. administering the polymer surfaced microcolloid to a mammal having damaged nervous system tissue.

2. The method of claim 1, wherein the damaged nervous system tissue is central nervous system tissue.

3. The method of claim 1, wherein the base material is selected from the group comprising silica or chitosan.

4. The method of claim 3, wherein the polymer surfaced microcolloid comprises individual spheres of approximately 5-400 nm in size.

5. The method of claim 5, wherein the individual spheres comprising the polymer surfaced microcolloid are approximately of the same size.

6. The method of claim 5, wherein the polymer is polyethylene glycol.

7. The method of claim 6, wherein the polymer surfaced microcolloid further comprises hydralazine.

8. A composition for treating a neural insult comprising:
a. a microcolloid having a base material selected from the group consisting of chitosan and a silicate;
b. an acrolein scavenger charged to load the microcolloid and operable to remove acrolein from the area of the neural insult when administered to an injured mammal.

9. The composition of claim 8, wherein the microcolloid is further surface coated with a membrane fusogen and the acrolein scavenger is contained within the microcolloid.

10. The composition of claim 9, wherein the membrane fusogen is polyethylene glycol and the acrolein scavenger is hydralazine.

11. The composition of claim 9, wherein the microcolloid base material is chitosan, and the acrolein scavenger is hydralazine.

12. The composition of claim 11, wherein the composition is operable to preferentially target damaged nerve tissue.

13. The composition of claim 11, wherein the composition operably reduces acrolein-mediated cell death when administered to a patient at lower dosage than hydralazine alone.

14. The composition of claim 8, wherein the neural insult is selected from the group consisting of an acute neural injury and a degenerative neural injury.

15. The composition of claim 8, wherein the microcolloid is produced by the combination of polyethylene glycol with tetramethyl orthosilicate by combining water-in-oil or reverse microemulsion with sol-gel technology.

16. The composition of claim 8, wherein the microcolloid is produced by the complexation of chitosan and dextran sulfate, where chitosan was dissolved in an acidic solution.

17. The composition of claim 8, where the microcolloid is produced by the complexation of chitosan and sodium tripolyphosphate in an acidic solution.

18. The composition of claim 17, wherein the acrolein scavenger is hydralazine.

19. The composition of claim 18, wherein the composition operably targets damaged cell membranes when administered to an animal suffering a neural insult.
